(19) United States
(12) Patent Application Publication
(10) Pub. No.: US 2012/0065470 A1
(43) Pub. Date: Mar. 15, 2012

(54) ROBOTIC SYSTEM TO AUGMENT ENDOSCOPES

(75) Inventors: Kevin C. Olds, Baltimore, MD (US); Russell H. Taylor, Severna Park, MD (US); Jeremy D. Richmon, Stevenson, MD (US)

(73) Assignee: The Johns Hopkins University, Baltimore, MD (US)

(21) Appl. No.: 13/232,617

(22) Filed: Sep. 14, 2011

Related U.S. Application Data
(60) Provisional application No. 61/382,557, filed on Sep. 14, 2010.

(51) Int. Cl. A61B 1/04; A61B 1/00

(52) U.S. Cl. 600/109; 600/118; 901/19; 901/27; 901/41

(57) ABSTRACT

A robotic system for steerable tip endoscopes includes a support arm, an endoscope gripping assembly rotatably connected to the support arm by a rotation assembly, and a translation assembly operatively connected to the support arm. The endoscope gripping assembly is configured to grip any one of a plurality of differently structured endoscopes, the translation assembly is configured to move the support arm along a linear direction to thereby move an endoscope when held by the endoscope gripping assembly along an axial direction, and the rotation assembly is configured to rotate the endoscope along a longitudinal axis of rotation.

### CROSS-REFERENCE OF RELATED APPLICATION

This application claims priority to U.S. Provisional Application No. 61/382,557 filed Sep. 14, 2010, the entire contents of which are hereby incorporated by reference.

### BACKGROUND

1. Field of Invention

The field of the currently claimed embodiments of this invention relates to robotic systems, and more particularly to robotic systems to augment endoscopes.

2. Discussion of Related Art

Many different types of operations require the use of a clinical endoscope, including laparoscopic surgery, many GI tract surgeries, many sinus surgeries, and trans-oral laryngeal and tongue-based surgeries (Iro et al. Minimally Invasive Surgery in Oto-Rhino Laryngology. European Archives of Oto-Rhino-Laryngology. Volume 250, Number 1, 1993; Vaughan, Charles et al. Laryngeal Carcinoma: Transoral Treatment Utilizing the CO2 Laser The American Journal of Surgery, Volume 136, Issue 4, October 1978, pp 490-493; Taylor, Russell et al. Computer Integrated Surgery Technology and Applications. pp 603-617. Boston Mass.: The MIT Press, 1996). These surgeries utilize two main types of endoscopes: flexible and rigid. When more direct access to the target area is possible, a rigid endoscope is normally used, but when such access is not possible, such as in GI tract surgery, or deep trans-oral laryngeal surgery, a flexible endoscope must be used. Robotic manipulation of rigid endoscopes is quite well developed, particularly for laparoscopic surgery with systems such as the Automated Endoscope System for Optimal Positioning (AESOP) and the DaVinci surgical system. There are two main approaches for robotically controlling an endoscope. The most common approach in the literature is to fully engineer a completely robotic endoscope from scratch, which provides excellent control, but is time consuming and expensive. The second approach is to build a robot to control a pre-existing clinical endoscope. The DaVinci system uses a custom endoscopic camera as part of its system, whereas AESOP manipulates a pre-existing rigid clinical endoscope (Taylor, Russell et al. Computer Integrated Surgery Technology and Applications. pp 577-580. Boston Mass.: The MIT Press, 1996; Taylor, Russell et al. Computer Integrated Surgery Technology and Applications. pp 581-592. Boston Mass.: The MIT Press, 1996; Horgan et al. Robots in Laparoscopic Surgery. Journal of Laparoendoscopic & Advanced Surgical Techniques; Volume 11, Number 6, 2001).

Robotic manipulation of flexible endoscopes, however, is far less developed, since they are inherently more difficult for a robot to control given their flexibility. There has been some work in robotic flexible endoscopes for GI tract surgery, but this has mainly involved complex custom engineered solutions rather than manipulation of clinical endoscopes (Taylor 1996 pp 577-580). One example of robotic manipulation of a clinical flexible endoscope is the pneumatic system proposed by Suzumori et al (Suzumori et al. New pneumatic rubber actuators to assist colonoscope insertion. Proceedings 2006 IEEE International Conference on Robotics and Automation. ICRA 2006). This system uses pneumatic actuators to assist in the insertion of a clinical colonoscope. This system is highly adapted to colonoscopy however, relying on contact friction against the colon walls to generate force. It also does not manipulate the endoscope body or end effector, since the pneumatic actuators only act on the flexible part of the endoscope shaft. Another approach was taken by Shin et al. for laparoscopic surgery (Shin et al. Design of a Dexterous and Compact Laparoscopic Assistant Robot. SICE-ICASE International Joint Conference 2006). They made a custom laparoscope consisting of a rigid shaft and a rigid end effector joined by a cable operated flexure. The body and end effector of the endoscope were then robotically controlled (Shin 2006).

A hand-held flexible endoscope manipulator is also known from Eckl et al. (Ekcl R. et al. Comparison of manual Steering and Steering via Joystick of a flexible Rhino Endoscope. 32nd Annual International Conference of the IEEE EMBS Buenos Aires, Argentina, Aug. 31-Sep. 4, 2010). Their system manipulates a flexible endoscope using a hand-held pistol-grip manipulator that controls scope rotation and tip angle, but not translation. They have also shown the ability to attach this system to a passive arm, and control it with a joystick. In both cases, this system lacks a translational motion degree of freedom, which makes full robotic operation impossible. There thus remains a need for improved robotic systems to augment control over endoscopes.

### SUMMARY

A robotic system for steerable tip endoscopes according to an embodiment of the current invention includes a support arm, an endoscope gripping assembly rotatably connected to the support arm by a rotation assembly, and a translation assembly operatively connected to the support arm. The endoscope gripping assembly is configured to grip any one of a plurality of differently structured endoscopes, the translation assembly is configured to move the support arm along a linear direction to thereby move an endoscope when held by the endoscope gripping assembly along an axial direction, and the rotation assembly is configured to rotate the endoscope along a longitudinal axis of rotation.

A robotically assisted or controllable flexible endoscope system according to an embodiment of the current invention includes a support arm, an endoscope gripping assembly rotatably connected to the support arm by a rotation assembly, a steerable tip endoscope held by a gripping mechanism of the endoscope gripping assembly, and a translation assembly operatively connected to the support arm. The translation assembly is configured to move the support arm along a linear direction to thereby move the endoscope, and the rotation assembly is configured to rotate the endoscope along a longitudinal axis of rotation.

### BRIEF DESCRIPTION OF THE DRAWINGS

Further objectives and advantages will become apparent from a consideration of the description, drawings, and examples.

FIG. 1 is an illustration of an example of a flexible endoscope that can be used with and/or incorporated as part of a robotic system according to embodiments of the current invention.

FIG. 2 shows an example of a robotic system for steerable tip endoscopes according to an embodiment of the current invention.

FIG. 3 shows another view a robotic system for steerable tip endoscopes according to an embodiment of the current invention.

FIG. 4 shows another view a robotic system for steerable tip endoscopes according to an embodiment of the current invention.

FIG. 5 shows another view a robotic system for steerable tip endoscopes according to an embodiment of the current invention.

FIG. 6 shows another view a robotic system for steerable tip endoscopes according to another embodiment of the current invention.

FIG. 7 shows a control unit that can be included in a robotic system for steerable tip endoscopes according to an embodiment of the current invention.

FIG. 8 shows a view of a rotation assembly and an endoscope tip control assembly for a robotic system for steerable tip endoscopes according to an embodiment of the current invention.

FIG. 9 shows water-tight covers for the rotation assembly and the endoscope tip control assembly of FIG. 8.

FIG. 10 shows a view of a translation assembly for a robotic system for steerable tip endoscopes according to an embodiment of the current invention.

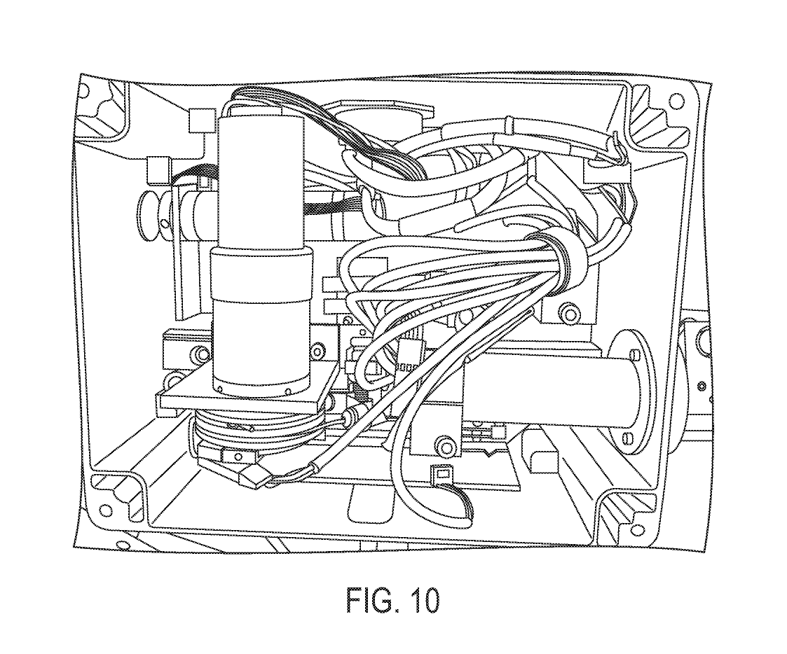

FIG. 11 shows a view of an electronics unit for a robotic system for steerable tip endoscopes according to an embodiment of the current invention.

FIG. 12 shows a view of a translation assembly for a robotic system for steerable tip endoscopes according to another embodiment of the current invention in which additional motor components are also included.

FIG. 13 shows a view of a rotation assembly for a robotic system for steerable tip endoscopes according to another embodiment of the current invention.

FIG. 14 shows a view of an endoscope tip control assembly for a robotic system for steerable tip endoscopes according to an embodiment of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Many clinical applications require the use of an endoscope with a flexible end effector, such as ablation of laryngeal tumors. Such operations are typically performed by two surgeons, with one surgeon using both hands to manipulate the endoscope, and another using a surgical laser and a tissue manipulation instrument. This results in a crowded operating room environment, and a need for significant coordination between two surgeons, thus increasing the difficulty and overall cost of the operation. Some embodiments of the current invention can solve these problems by using a robotic system to manipulate the endoscope. Since modern clinical endoscopes have a working channel that a laser fiber can pass through, the laser can also be incorporated into the endoscope. The robotic system can allow single handed operation of the endoscope with the laser inside, allowing one surgeon to perform the entire operation using one hand to manipulate the endoscope/laser and one to use a tissue manipulation instrument. Since the weight of the endoscope and the force needed to manipulate the handle can both be handled by the robot, surgeon fatigue can be reduced as well.

The robot can hold the endoscope in a fixed position or precisely move each degree of freedom with virtually no tremor, thus improving surgical accuracy. Since the endoscope outputs a digital video signal, it is also possible for the robot to utilize this to provide more advanced features, such as image stabilization, 3D reconstruction from endoscopic images using endoscope motions to create a stereo baseline, image overlay of relevant data on the endoscope video feed, detailed recording of the endoscope motions used in a surgical procedure, which could later be used for training or position recall, and virtual fixtures for added safety.

Some embodiments of the current invention can provide a compact, sterilizable, robust, accurate, robotic system for operating an unmodified clinical flexible endoscope with one hand. This can reduce the number of personnel needed to perform many operations, and also can keep the endoscope in position if the surgeon needs to release it to perform another task. The introduction of a robotic system between the surgeon and the endoscope can also increase accuracy, since hand tremor can be largely eliminated. By robotically supporting and manipulating the endoscope, surgeon fatigue can be reduced as well. Also a rigid endoscope rather than a flexible one can be used, if desired.

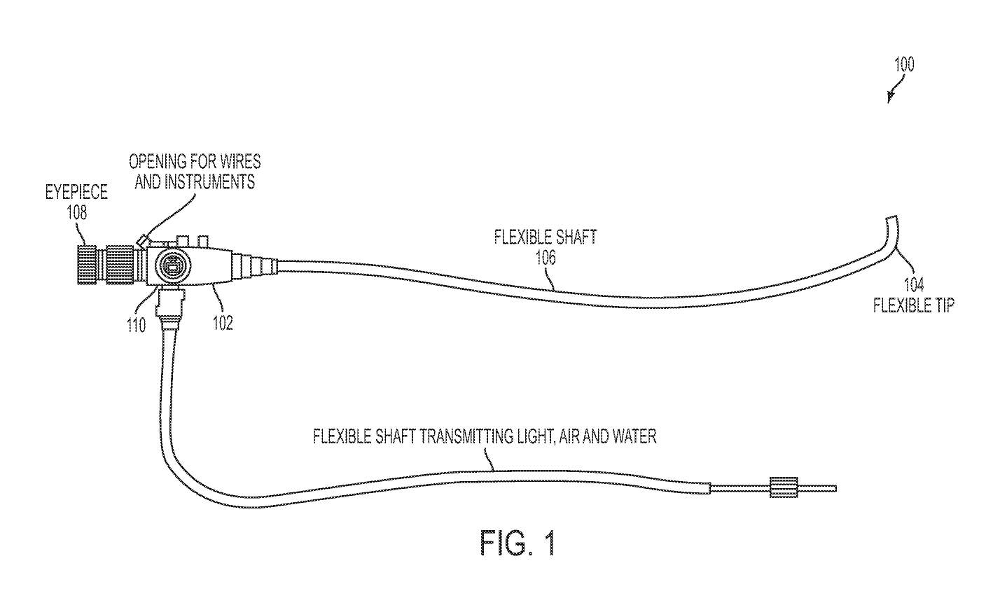

FIG. 1 is an illustration of an example of a flexible endoscope 100 that can be used with or incorporated into embodiments of the current invention. The flexible endoscope 100 can be, but is not limited to, a conventional hand-held flexible endoscope. The flexible endoscope can be, for example, a laryngoscope, a colonoscope, a bronchoscope, or any of a variety of flexible endoscopes. The endoscope 100 has a hand piece 102 at a proximal end and a flexible tip 104 at a distal end of the flexible endoscope 100. The endoscope 100 also has a flexible shaft 106 and an eyepiece 108. The eyepiece 108 can be used for direct viewing by an observer, or can be attached to an image pickup device, such as a video camera, for example. In this example, the flexible endoscope 100 has a knob 110 that can be used manually to control the flexible tip 104.

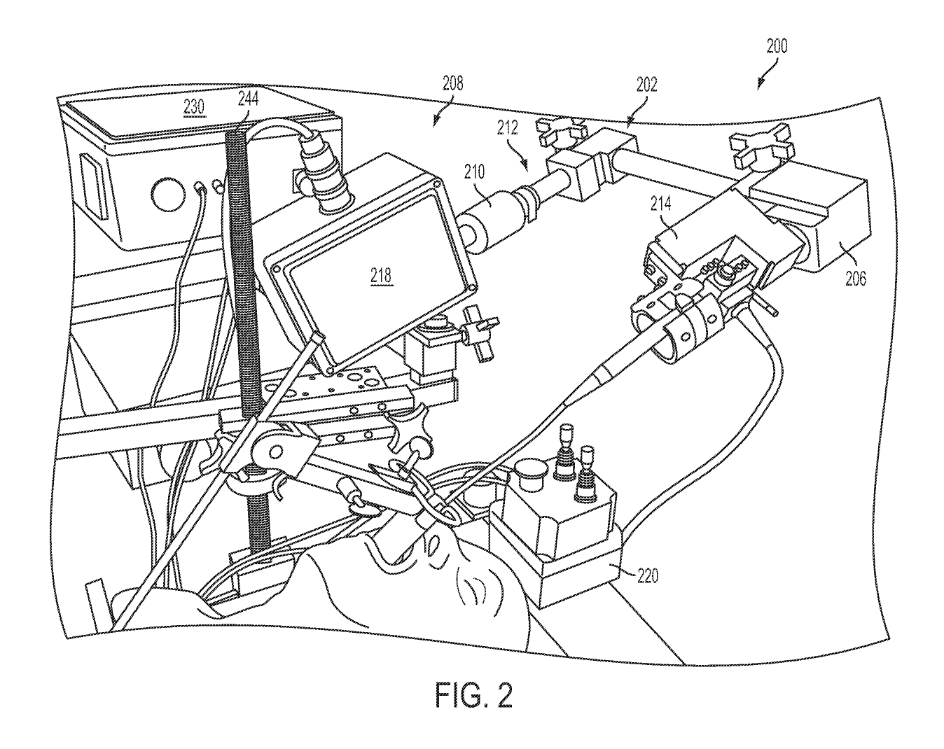

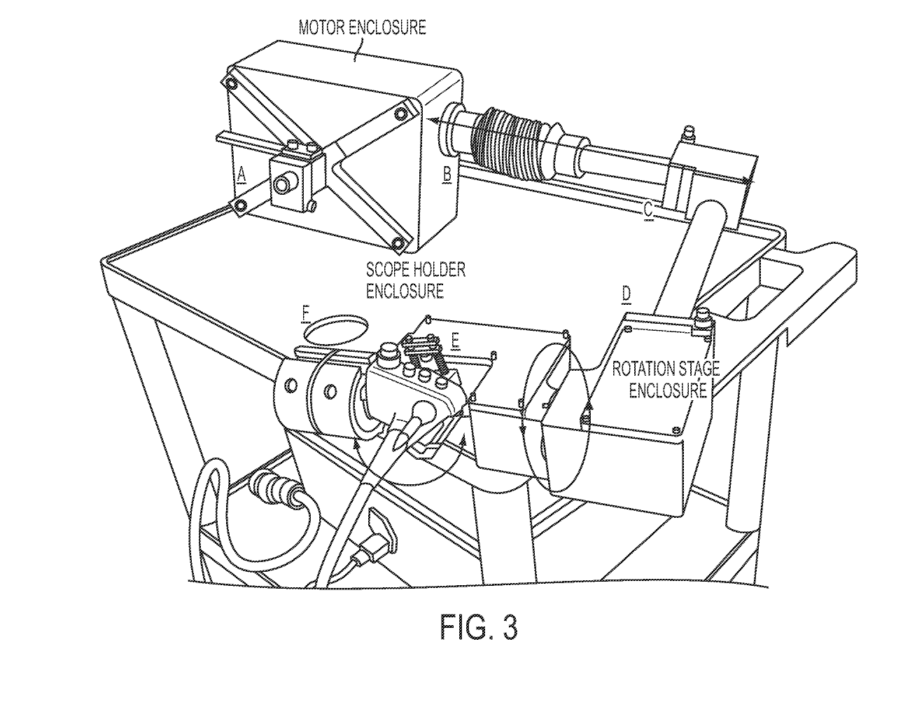

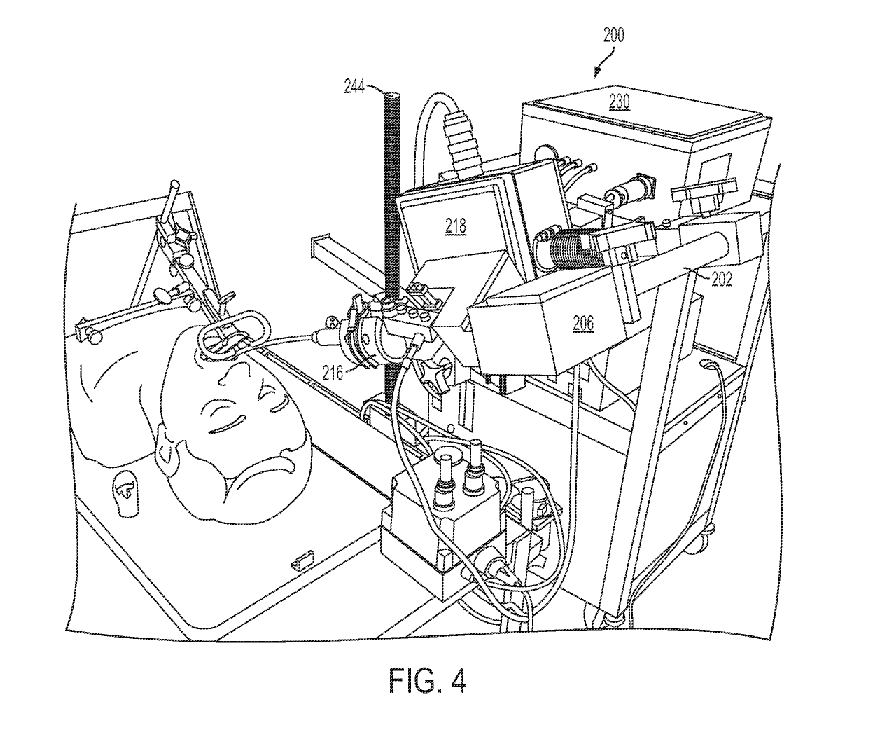

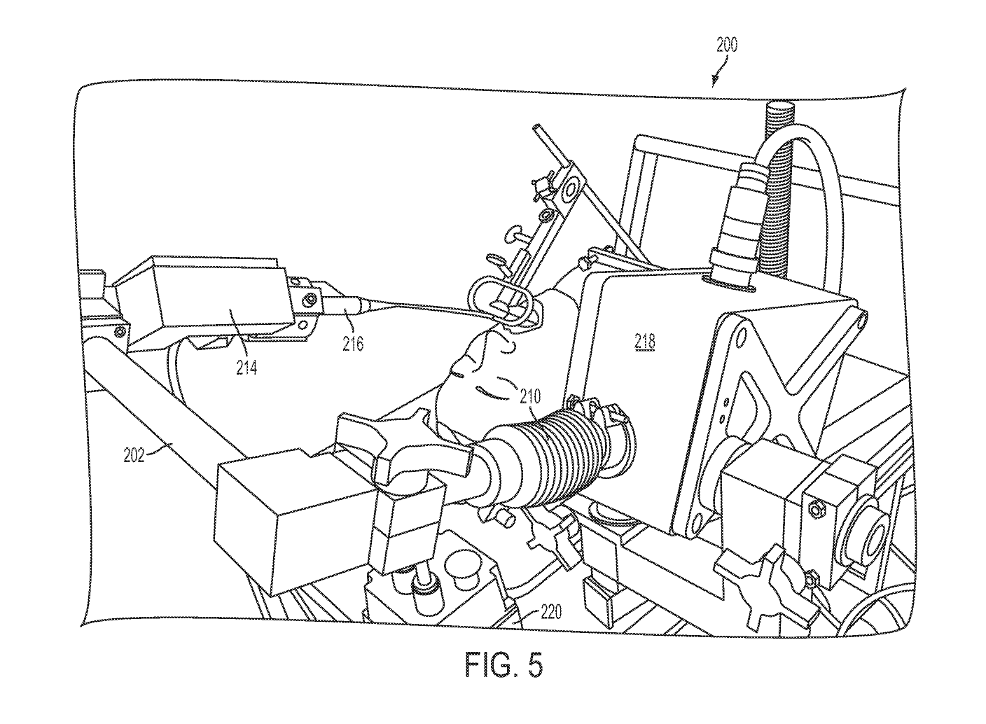

FIG. 2 shows an embodiment of a robotic system 200 for steerable tip endoscopes according to an embodiment of the current invention. FIGS. 3-6 show additional views of the robotic system 200. The robotic system 200 includes a support arm 202, an endoscope gripping assembly 204 rotatably connected to the support arm 202 by a rotation assembly 206, and a translation assembly 208 operatively connected to the support arm 202. The endoscope gripping assembly 204 is configured to grip any one of a plurality of differently structured endoscopes. The translation assembly 208 is configured to move the support arm 202 along a linear direction to thereby move an endoscope when held by said endoscope gripping assembly 204 along an axial direction. The rotation assembly 206 is configured to rotate the endoscope along a longitudinal axis of rotation.

In the embodiment of FIG. 2, a bellows 210 encloses a moveable section of the translation assembly 208 to keep it water proof to facilitate cleaning and sterilization. The support arm 202 is articulated with a straight segment 212 that moves linearly in response to operation of the translation assembly 208.

The robotic system 200 also includes an endoscope tip control assembly 214 adapted to be attached to the endoscope 216 to permit control of a flexible tip of the endoscope 216. Portions of the translation assembly 208 as well as electronics are contained within the waterproof box 218.

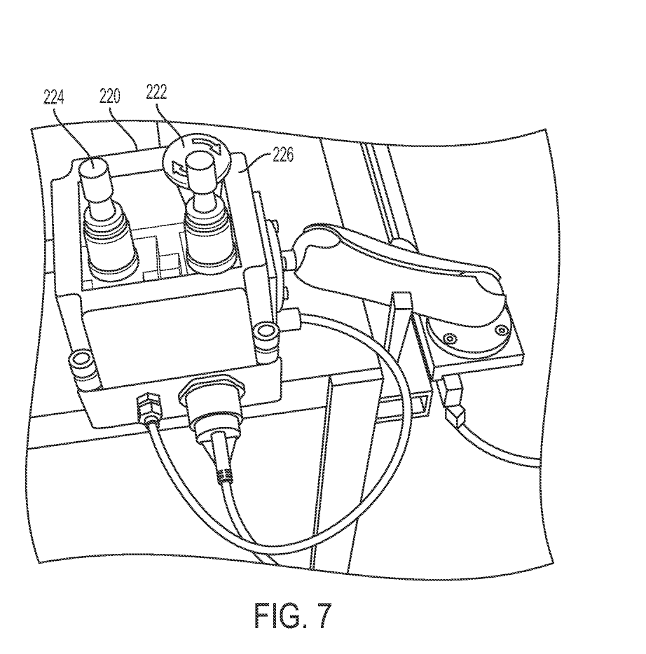

The robotic system 200 can also include a control unit 220 to allow a user to directly control at least one of the translation assembly 208, the rotation assembly 206 or the endoscope tip control assembly 214. FIG. 7 shows a more detailed view of an embodiment of the control unit 220. In this example, the control unit 220 has an emergency shut off switch 222, a two-dimensional joystick 224 and a one-dimensional joystick 226.

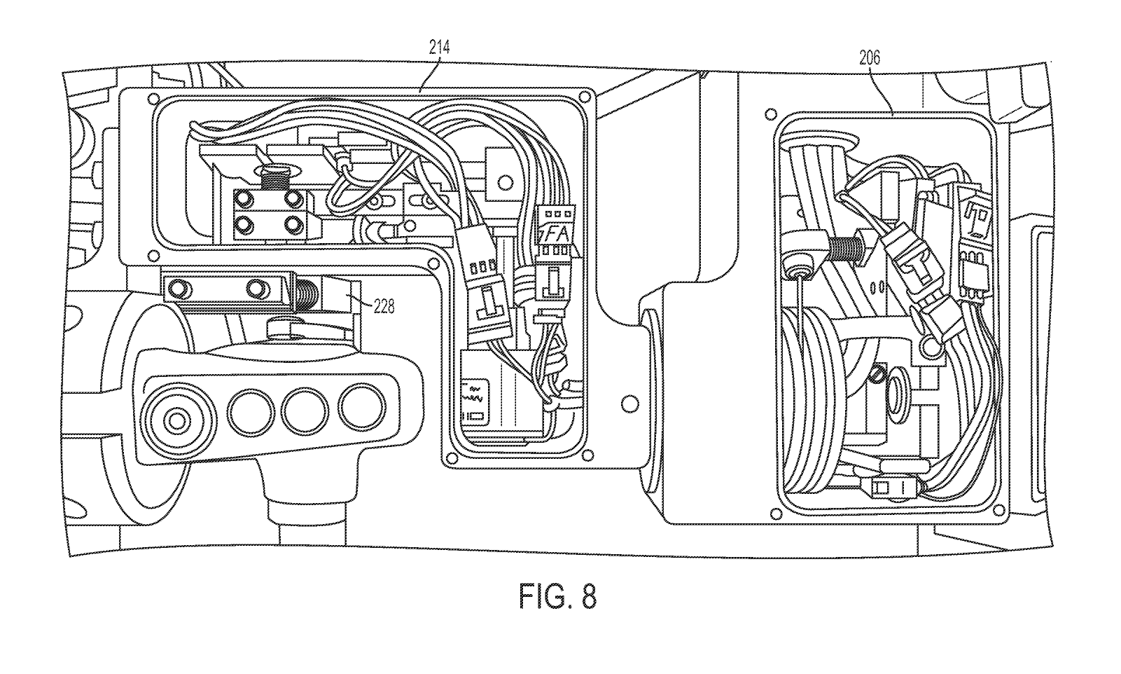

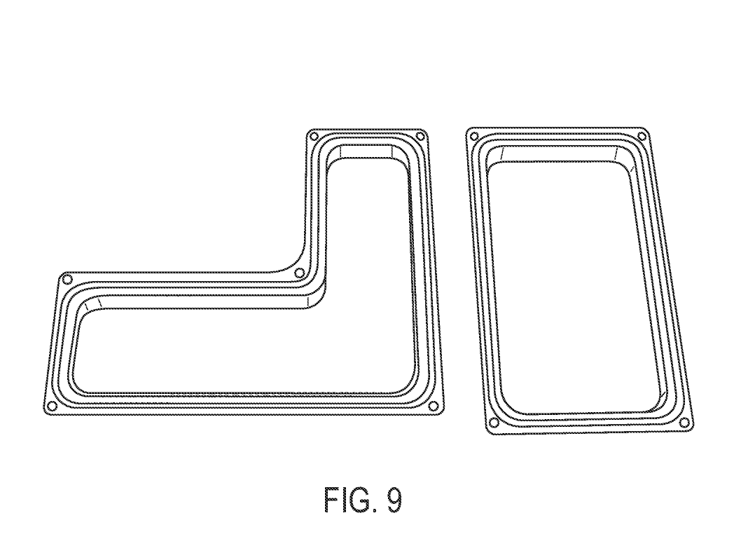

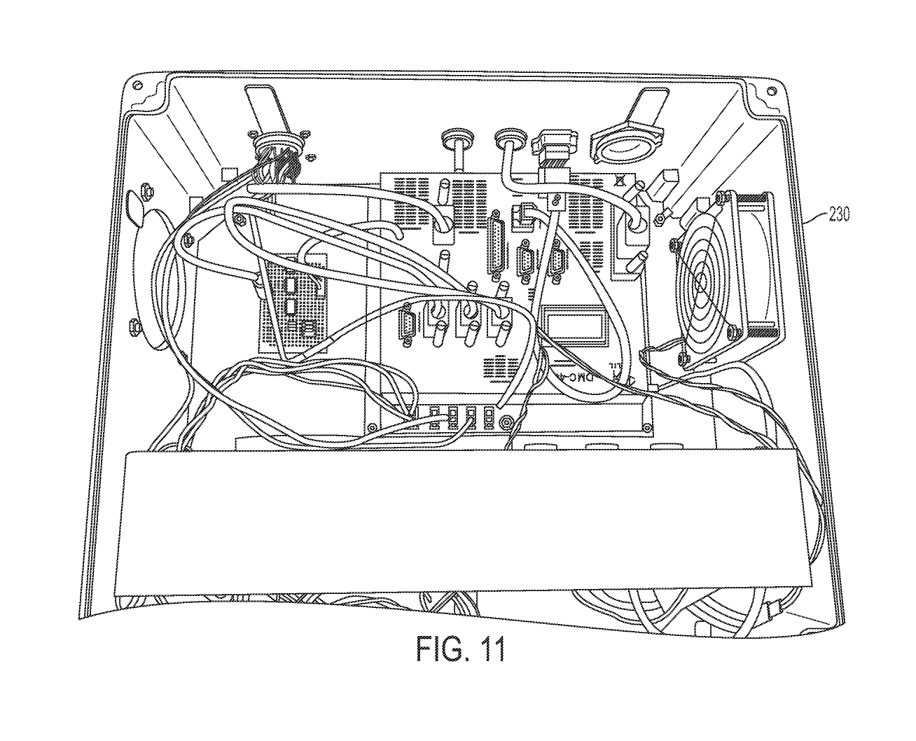

FIGS. 2-6 of the robotic system 200 show the translation assembly 208, the rotation assembly 206 and the endoscope tip control assembly 214 contained within water-tight enclosures to facilitate cleaning and sterilization for surgical use. In some embodiments, components of the assemblies can be localized within a single containment structure, or they could have components distributed over containment structures. FIG. 8 shows an example in which the endoscope-tip control assembly 214 and the rotation assembly 206 have electric motors that are fully contained within the respective structures. The structures are open in the view of FIG. 8 to show the interior components. FIG. 9 shows corresponding covers that can include o-rings for sealing the containments structures such that they are water tight. The endoscope tip control assembly 214 also includes a spring actuated clamp 228 to clamp on to the control knob for the flexible tip of the endoscope so that it can be turned by the endoscope tip control assembly 214. FIG. 19 shows the interior of the containment structure for the translation assembly 208 in which an electric motor drives a screw component. FIG. 11 shows the interior of the electronics container with the top open.

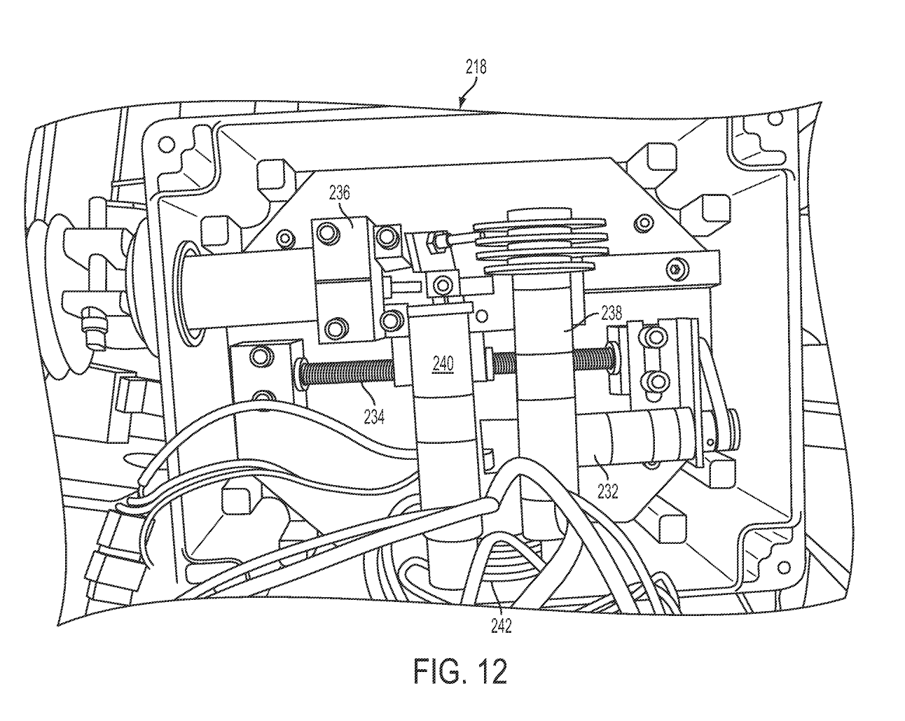

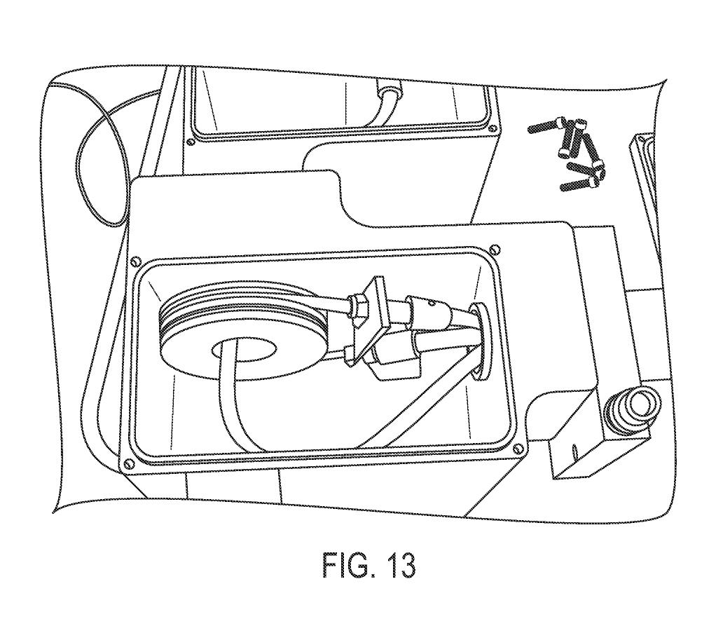

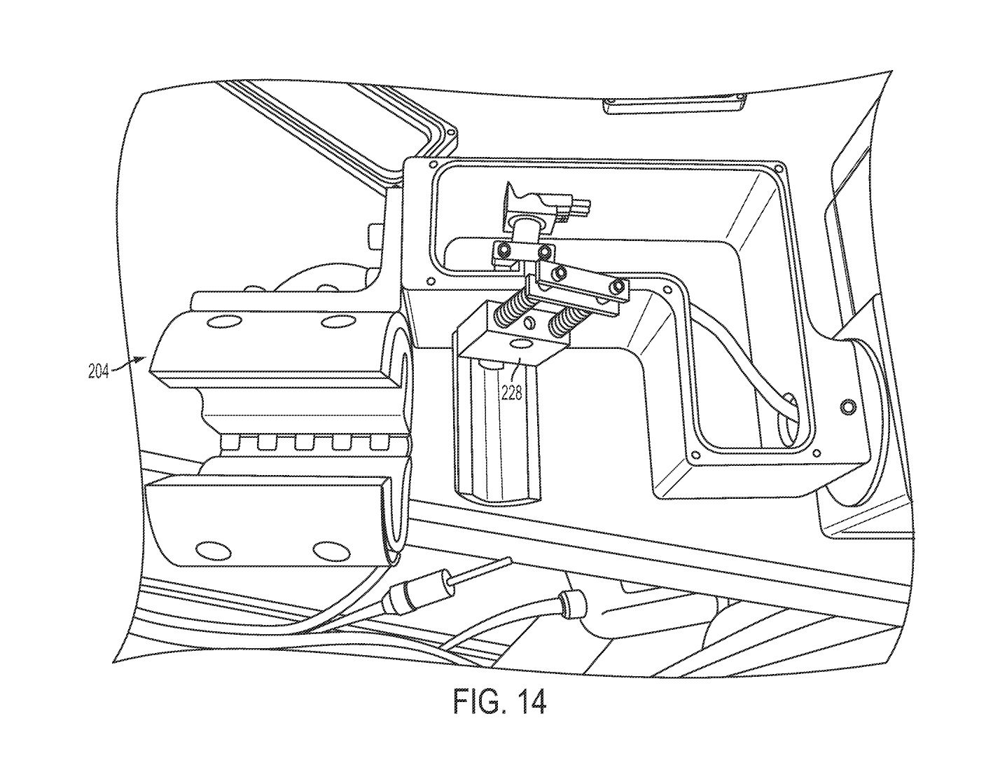

FIG. 12 shows an alternative embodiment of the interior of the motor enclosure 218 that contains translation assembly 208 as well as motors for the rotation assembly 206 and the endoscope tip control assembly 214. In this embodiment, a motor 232 drives translation stage with a belt connected to the screw 234 for the linear guide block and rail assembly for translation stage 236. An Acme screw is suitable for screw 234 in some embodiments. Motor 238 drives the rotation stage via a pulley and Bowden cables. Motor 240 drives the distal tip control knob. A waterproof connector 242 is provided for all electrical connections. FIGS. 13 and 14 show the interiors of the rotation assembly 206 and endoscope tip control assembly 214 corresponding to the embodiment of FIG. 12 in which Bowden cables run through the support arm 202.

The robotic system 200 can also include an image pickup system connected to the endoscope gripping assembly 204 according to some embodiments such that the image pickup system can be rotated by the rotation assembly 206 along with the endoscope 216. The image pickup system can be, but is not limited to, a video camera.

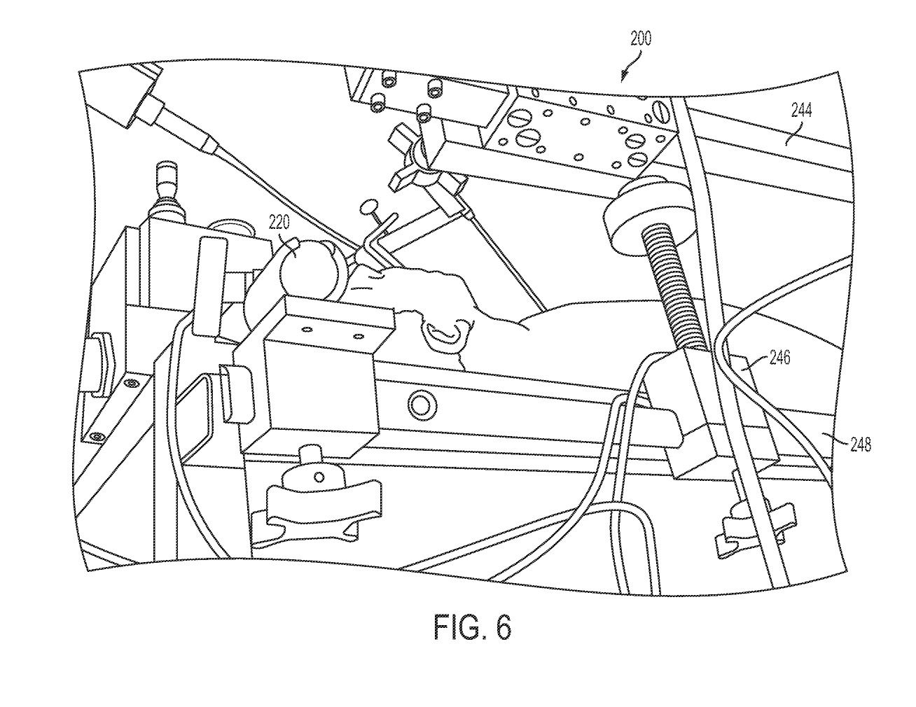

The robotic system 200 can also include a support frame 244 in some embodiments that is adapted to hold the support arm 202. The support frame can be a free-standing support frame, or can be adapted to mount to another structure. In the embodiment of FIG. 6, the support frame 244 has a bedrail mount 246 such that the robotic system 200 can be attached to a bedrail 248. In this example, the control unit 220 is also mounted to the bedrail 248 with a bedrail mount 250.

In operation, the robotic system 200 can be fully autonomous, remotely operated, or locally operated, for example by the use of control unit 220. The robotic system can be placed into rough proximity to where it will be used. The translation assembly 208 moves the section 212 of the support arm 202 back and/or forth in a linear direction. This translates the endoscope 216 back and forth along a linear direction to extend more or less along the path of interest. The rotation assembly 206 rotates the body of the endoscope 216 similar to how one would rotate the body of an endoscope by hand. The endoscope tip control assembly 214, which is connected to the control knob of the endoscope 216, rotates the control knob back and/or forward to effect motion of the flexible tip of the endoscope.

The embodiments shown above have three degrees of control, i.e., translation of the endoscope along a linear path, rotation of the endoscope about an axis of the endoscope handle, and control of the flexible tip of the endoscope. Other embodiments could include robotic and/or robot assisted control of additional degrees of freedom, if desired.

### Example 1

A prototype was constructed using an old clinical laryngoscope and the Laparoscopic Assisted Robotic System (LARS) robot from the Laboratory for Computational Sensing and Robotics at Johns Hopkins University, augmented in the following ways:
The scope was attached to the LARS using a custom made adaptor
The scope's handle was controlled using a custom made linkage and servo motor system attached to the adaptor
Custom electrical systems were added to control this extra motor
The program of the LARS was heavily modified to integrate these additional systems, and also to change its behavior to be appropriate for this application

The prototype was successfully tested using a rubber airway phantom.

Three degrees of freedom are often desired for the robotic control of the flexible laryngoscope; one to translate the endoscope in and out of the airway, one to rotate the endoscope along its axis through the airway, and one to control the tip of the endoscope.

A plastic (delrin) adaptor was machined to securely hold the endoscope and interface it to the LARS. We chose a servo motor to control the scope handle, and machined aluminum brackets to attach the motor to the adaptor. To interface the motor to the scope handle we considered both a timing belt system and a 4 bar linkage, and chose the latter for simplicity and adjustability. The linkage was machined from aluminum. Since the endoscope requires an external camera, and the camera was not rotationally fixed to the scope, we designed and fabricated an aluminum camera holder to keep the camera fixed with respect to the scope. We also machined an aluminum bracket to hold the connector for the motor wiring to reduce strain on the motor wires.

An additional microprocessor was added to the LARS robot since it was not able to control an additional servo necessary for scope function. For power, we tapped into the 12V DC supply of the LARS, and used a switching voltage regulator to achieve the 5V needed for the servo. Since this type of servo does not have position feedback we custom modified it to provide one by tapping into its internal position feedback. Since this signal is very noisy due to sharing its ground with the servo motor's power ground, we added a buffered low-pass filter before passing the signal to the microprocessor's A/D converter. The microprocessor and associated electronics are all contained in their own enclosure which is not coupled to the scope.

The LARS robot software was modified to adapt it to the novel task. We used a 3D space mouse to control the LARS two degrees of freedom as well as the scope tip movement. The complicated dynamics of the scope tip motion relative to the handle motion can lead to problems. The tip motion is both highly nonlinear and exhibits significant hysteresis. Hysteresis compensation was added to the software to compensate for this.

We tested the finished prototype with a phantom airway model. We were able to freely navigate the inside of the airway using the prototype. Notable improvements in stability and accuracy over using the scope by hand were achieved.

### Example 2

A fully functional robotically-controlled distal-tip flexible laryngoscope that meets the appropriate safety standards for operating room use was constructed. This embodiment includes some or all of the following features:
Robot is fully enclosed and sealed, making it suitable for wash-down applications.
Robot is designed to mount easily to a Chung retractor for easy attachment to a surgical bed.
Robot uses an easily changeable molded rubber adaptor to hold the endoscope, and an adjustable spring-loaded manipulator to control the endoscope handle, making it easy to use different models of endoscope.
No modifications to the endoscope are necessary since the endoscope handle manipulator simply cradles the endoscope handle.
Robot has adjustable joints which allow the surgeon to configure it as needed.
Robot's main body is over the side of the bed, thus minimizing the amount of weight and bulk over the patient.
Robot can include an adjustable malleable support for the flexible shaft of the endoscope to prevent it from drooping.

One embodiment used a Bowden cable mechanism to move the scope handle manipulator with the driving motor in the motor enclosure. In other embodiments, these can be replaced by a motor and linkage placed directly in the endoscope holder enclosure. The rotation of the endoscope is achieved via a Bowden cable pulley system actuated by a motor in the motor enclosure.

### Adjustable Malleable Endoscope Support:

The robot can also include an adjustable malleable support for the un-actuated flexible portion of the endoscope. This can allow the surgeon to bend the endoscope roughly into a desired configuration, and then manipulate it with the robot essentially as though the shaft were rigid. The support can include a bendable metal wire encased in medical grade rubber tubing, for example, which can be fixed to the endoscope shaft either by wrapping it around the shaft, or connecting it with surgical rubber loops. A surgical rubber casing can protect the patient from direct exposure to the aluminum support wires.

Further electrical and mechanical modifications to the system can include:
An extensive passive positioning system allowing the robot to be attached to a surgical bed rail and easily adjusted.
Improved electronics including filters for all sensors, and a computer-controlled relay for emergency shut-off.
A custom joystick system which also attaches to the bedrail.
Friction collars on necessary passive joints to prevent them from moving suddenly when unlocked.
Motor upgrades to provide improved performance.
Preliminary integrated computer vision guidance utilizing the video stream from the scope.
Quick-release latch for scope holder.

The new custom passive positioning system not only allows the surgeon to adjust the position of the endoscope, but also to easily insert and remove the endoscope. The robot's insertion/extraction degree of freedom only has about 3.5 inches of motion in this example, so the 18 inch horizontal motion of the passive positioning arm allows the surgeon to coarsely insert the scope to the desired location, and then manipulate it with precision using the robotic degrees of freedom. All of the passive degrees of freedom are also lockable, to prevent undesired motion when the surgeon is operating. The two passive degrees of freedom that present a risk of moving independently under the force of gravity when unlocked have been fitted with friction collars to prevent any sudden inadvertent motion. All passive degrees of freedom can be locked and unlocked using a knob, which allows for quick adjustment. In addition, the robot's elbow joint can be used to quickly raise the scope away from the patient's head in case of emergency.

The custom joystick system can be mounted directly to the bed rail, eliminating the need for extra tables or bed space for a conventional joystick, and also eliminating the chance slippage or of dropping a conventional joystick and thus giving false commands to the robot. The joystick enclosure's position can also be adjustable, using a lockable passive positioning arm. The joystick enclosure also incorporates an emergency off switch in this example, which physically cuts the power to all the motors, and a USB controlled relay, which allows the robot control computer to shut off the motor power if any faults are detected. The whole joystick assembly uses corrosion resistant, non-toxic, water-tight components, so it is wash-down compatible.

An embodiment of this invention is a three degree of freedom robot as described above which actuates both the body and flexible end effector of an unmodified clinical endoscope, with a malleable support for the scope shaft. It would also be possible to add extra degrees of freedom if desired, though three is all that is necessary to achieve many specific tasks with minimum complexity. Embodiments of the current invention can be useful for laryngeal surgery, for example. However, the broad concepts of the current invention are not limited to this example. Other embodiments can be applied for a colonoscope, a bronchoscope, or any of a variety of flexible or rigid endoscopes.

Though the prototypes were constructed mostly from aluminum, other materials can be used. For example, injection molded plastic parts may be suitable in many applications. It is also possible to mount motors directly at all of the joints rather than using cables to transmit mechanical forces. It is also be possible to mount the robot independently of the surgical bed, if desired. However, this is often not desirable because relative motion between the robot and the bed can degrade the endoscope image quality.

The system could also be implemented as a hand-held device that can be detachable from the translation stage. This would allow the surgeon to operate the device hand-held when convenient, and then attach the handheld component to the translation stage for more precise operation.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A robotic system for steerable tip endoscopes, comprising:
a support arm;
an endoscope gripping assembly rotatably connected to said support arm by a rotation assembly; and
a translation assembly operatively connected to said support arm,
wherein said endoscope gripping assembly is configured to grip any one of a plurality of differently structured endoscopes,
wherein said translation assembly is configured to move said support arm along a linear direction to thereby move an endoscope when held by said endoscope gripping assembly along an axial direction, and
wherein said rotation assembly is configured to rotate said endoscope along a longitudinal axis of rotation.

2. A robotic system for endoscopes according to claim 1, further comprising an endoscope tip control assembly adapted to be attached to said endoscope to permit control of a flexible tip of said endoscope.

3. A robotic system for endoscopes according to claim 1, wherein said support arm is an articulated support arm.

4. A robotic system for endoscopes according to claim 2, further comprising a control unit to allow a user to directly control at least one of said translation assembly, said rotation assembly or said endoscope tip control assembly.

5. A robotic system for endoscopes according to claim 2, wherein said translation assembly, said rotation assembly or said endoscope tip control assembly are all contained within water-tight enclosure to facilitate cleaning and sterilization for surgical use.

6. A robotic system for endoscopes according to claim 1, further comprising an image pickup system connected to said endoscope gripping assembly such that said image pickup system is rotated by said rotation assembly along with said endoscope.

7. A robotic system for endoscopes according to claim 6, wherein said image pickup system is a video camera.

8. A robotic system for endoscopes according to claim 1, further comprising a support frame adapted to hold said support arm.

9. A robotic system for endoscopes according to claim 8, wherein said support frame is a free-standing support frame.

10. A robotic system for endoscopes according to claim 8, wherein said support frame comprises a bedrail mount such that said robotic system for endoscopes can be attached to a bedrail.

11. A robotically assisted or controllable flexible endoscope system, comprising:
a support arm;
an endoscope gripping assembly rotatably connected to said support arm by a rotation assembly;
a steerable tip endoscope held by a gripping mechanism of said endoscope gripping assembly; and
a translation assembly operatively connected to said support arm,
wherein said translation assembly is configured to move said support arm along a linear direction to thereby move said endoscope, and
wherein said rotation assembly is configured to rotate said endoscope along a longitudinal axis of rotation.

12. A robotically assisted or controllable endoscope according to claim 11, wherein said endoscope is a flexible endoscope.

13. A robotically assisted or controllable endoscope according to claim 11, further comprising an endoscope tip control assembly adapted to be attached to said endoscope to permit control of a flexible tip of said endoscope.

14. A robotically assisted or controllable endoscope according to claim 11, wherein said support arm is an articulated support arm.

15. A robotically assisted or controllable endoscope according to claim 13, further comprising a control unit to allow a user to directly control at least one of said translation assembly, said rotation assembly or said endoscope tip control assembly.

16. A robotically assisted or controllable endoscope according to claim 13, wherein said translation assembly, said rotation assembly or said endoscope tip control assembly are all contained within water-tight enclosures to facilitate cleaning and sterilization for surgical use.

17. A robotically assisted or controllable endoscope according to claim 11, further comprising an image pickup system connected to said endoscope gripping assembly such that said image pickup system is rotated by said rotation assembly along with said endoscope.

18. A robotically assisted or controllable endoscope according to claim 11, further comprising a support frame adapted to hold said support arm.

19. A robotically assisted or controllable endoscope according to claim 18, wherein said support frame is a free-standing support frame.

20. A robotically assisted or controllable endoscope according to claim 18, wherein said support frame comprises a bedrail mount such that said robotic system for endoscopes can be attached to a bedrail.
